(19) United States
(12) Patent Application Publication
(10) Pub. No.: US 2013/0158048 A1
(43) Pub. Date: Jun. 20, 2013

(54) Formulation Solution Adapted to Prolong Plasma Times of Drugs in Mammals Including Humans

(71) Applicants: Aida Salatinjants, Los Angeles, CA (US); Robert Salantinjants, Los Angeles, CA (US)

(72) Inventors: Aida Salatinjants, Los Angeles, CA (US); Robert Salantinjants, Los Angeles, CA (US)

(21) Appl. No.: 13/709,381

(22) Filed: Dec. 10, 2012

Related U.S. Application Data
(63) Continuation of application No. 13/330,470, filed on Dec. 19, 2011.

(51) Int. Cl. A61K 47/24

(52) U.S. Cl. CPC A61K 47/24
USPC 514/254.11

(57) ABSTRACT

In the method of administering rifampicin to a human subject, the improvement wherein a prolongation composition is also administered, said prolongation composition comprising:

| Pectin | 0.94 | g |
|---|---|---|
| Potassium bitartrate | 0.48 | g |
| Tannic acid | 0.8 | g |
| L-Tyrosine | 0.21 | g |
| Riboflavin | 2 ml 10% solution of 15% Ethyl Alcohol | |
| Hexanoic Acid (Caproic acid) | 0.06 | ml |

| | |
|---|---|
| Ethyl Alcohol | 15% Ethyl Alcohol to make 100 ml |

This is a continuation of pending application Ser. No. 13/330,470 filed Dec. 19, 2011, the disclosures of which are incorporated herein by reference.

### BACKGROUND OF THE INVENTION

It is known that the clinical pharmacology of the sulfonamides is generally characterized by the following. A single 2.0 gram dose of sulfisoxazole results in a mean time of peak plasma concentration of 2.5 hours. About 97% of the original dose is excreted in the urine within 48 hours. The mean elimination half life is 5.8 hours, ranging from 4.6 to 7.8 hours. These characteristics are typical of the other “sulfa”-type drugs such as sulfamethizole, sulfamethoxazole and sulfasalazine.

In the case of quinine, its salts, and the other cinchona alkaloids, peak plasma concentrations occur within 1 to 3 hours after a single oral dose of 260 mg in the form of the sulfate. The half-life is 4 to 5 hours. After termination of quinine therapy, the plasma level falls rapidly and is barely detectable after 24 hours.

U.S. Pat. Nos. 4,708,952 and 4,716,173 disclose an aqueous prolongation composition adapted to prolong the residence time of sulfa and cinchona alkaloid drugs in the circulating plasma of mammals including humans comprising the hexanoic acid, potassium hydrogen tartrate, tannic acid pectin and riboflavin, with the further presence of glutamic acid in the case of sulfa drugs and L-tyrosine in the case of the cinchona alkaloids, a total of six ingredients.

The present invention directly increases the half-life, efficacy and pharmacological activity of various medications (cinchona alkaloids, sulfanamides, primaquinine, decoquinate, rifampicin, verapamil, glipizide papaverin, streptomycin and others), which thereby provides more uniform plasma levels and increases the effect of active drugs. As a result, the dosing-requirement of the active drug is greatly reduced. The efficacy is so dramatic, that only one-time dosing regiment may be required, thus there is an associated decrease in the number and frequency of the side effects of drug.

### SUMMARY OF THE INVENTION

### Example 1

The composition adapted to prolong the residence time of drugs in the circulating plasma of mammals including humans comprises the following:

| Pectin | 0.94 | g |
|---|---|---|
| Potassium bitartrate | 0.48 | g |
| Tannic acid | 0.8 | g |
| L-Tyrosine | 0.21 | g |
| Riboflavin | 2 ml 10% solution of 15% Ethyl Alcohol | |
| Hexanoic Acid (Caproic acid) | 0.06 | ml |

| | |
|---|---|
| Ethyl Alcohol | 15% Ethyl Alcohol to make 100 ml |

### Example 2

The composition adapted to prolong the residence time of drugs such as the sulfa drugs in the circulating plasma of mammals including humans comprises the following:

| Pectin | 0.94 | g |
|---|---|---|
| Potassium bitartrate | 0.48 | g |
| Tannic acid | 0.8 | g |
| Glutamic acid | 0.21 | g |
| Riboflavin | 2 ml 10% solution of 15% Ethyl Alcohol | |
| Hexanoic Acid (Caproic acid) | 0.06 | ml |

| | |
|---|---|
| Ethyl Alcohol | 15% Ethyl Alcohol to make 100 ml |

The above figures are approximations on the order of 0±10%.

This prolongation composition uses 15% ethyl alcohol (ethanol) as the solvent. The ethyl alcohol must be present in at least a sufficient amount to dissolve the other ingredients. It has been found that the use of 15% ethyl alcohol and 0.8 g of tannic acid in lieu of 0.08 g of tannic acid as in the prior art alters the uptake and retention of the drug in mammals by producing a more uniform serum and organ level over time.

### DESCRIPTION OF THE PREFERRED EMBODIMENTS

The effective amount of the prolongation composition of this invention is based on body weight of the patient with special doses for active drug. The basic use of the compositions is to administer it at the time of administering the active drug. The ingredients used in the prolongation compositions are natural chemical ingredients and readily available and known in the medical profession, however it is the combinations and concentration which are critical.

The following Examples demonstrate the prolongation effect. The prolongation effect is not limited to the drugs presented in the Examples.

### Examples

The following examples used laboratory rats and mice.

### Example 3

Decoquinate solution was mixed with two solutions: one with the prolongation solution of Example 1 and one without the prolongation solution. Each solution contained an identical and normal dosage of the drug. In both cases, 0.5 ml of solution was injected into mice of approximately identical weight. The concentration of the drug in plasma and liver was chected by high performance then layer chromatography in mouse liver and plasma. The concentration of drug in plasma and liver using the prolongation solution did not change for eight hours, compared to the control which had no prolongation solution as shown in the following table:

| | PLASMA | | LIVER | |
|---|---|---|---|---|
| Hours | NE | PF | NE | PF |
| 0.5 | 25565 | 15213 | 313860 | 149675 |
| 1 | 49911 | 24638 | 430820 | 223565 |
| 2 | 43941 | 27350 | 317950 | 191725 |
| 4 | 35427 | 29216 | 323180 | 219615 |
| 8 | 25131 | 27882 | 177470 | 181015 |
| 24 | 697 | 457 | 7014 | 5549 |

NE-No Prolongation Solution
PF-Prolongation Solution

As these data show, there was relatively little change between 1 and 8 hours in the level of Decoquinate when the prolongation solution was used. When no prolongation solution was used, the Decoquinate level dropped between 1 and 8 hours by 50% in plasma and by almost as much in liver.

### Example 4

Rifampicin (Rifampin) was mixed with prolongation solution of Example 1 in proportion about 18 mg of Rifampicin in 3 ml of prolongation solution. Serum testing was conducted after one-time initial administration to rats. The Rifampicin level remained:

at 14 Hours-19 mcg/ml at 32 Hours-3.5 mcg/ml

Rifampicin was mixed with prolongation solution in proportion about 8 mg/rat. Result:

at 14 hours-14 mcg/ml

at 32 Hours-8.5 mcg/ml

Control test using water alone on 8 mg/rat Rifampicin resulted in the drug not being detected after 14 hours. Thus, the prolongation solution is effective at reduced drug administration levels.

Peak plasma levels of Rifampicin following a single 600 mg dose (human) range from approximately 3-13 mcg/ml at approximately 1.8 hours post dose. In healthy adults, the mean biological half-life of Rifampicin in serum between 3-5 Hours after 600 mg dose.

### Example 5

Verapamil HCL 9 mg was mixed with 3 ml of prolongation solution of Example 1 and injected into rats. Serum testing was conducted after one-time initial administration, the Verapamil HCL level remained:

at 2 hours-170 ng/ml

at 24 hours-190 ng/ml

at 24 hours-140 ng/ml

at 24 hours-240 ng/ml

Control test using water alone on 9 mg/rat Verapamil HCL resulted in none being detected after 24 hours.

Probable therapeutic range 70-350 ng/ml. Peak plasma concentration of verapamil (human) between 1 and 2 hours and mean elimination half-life ranged from 4,5 to 12 hours.

### Example 6

Glipizide was mixed with the prolongation solution of Example 1 in proportion about 0.25 mg of glipizide in 3 ml of prolongation solution, and injected into rats. Serum testing was conducted after one-time initial administration. Glipizide levels remained:

at 26 hours-0.57 mcg/ml

at 52 hours-1.4 mcg/ml

Peak serum level of glipizide following a single 5 mg dose: 0.1-0.5 mcg/ml at 1.6 hours post dose, biological half-life in serum between 2-5 hours.

### Example 7

Quinine 0.032 g was dissolved in 3 ml of the prolongation solution of Example 1 and injected into rat. Serum testing was conducted after one-time initial administration. The Quinine levels remained:

at 45 min.-4600 ng/ml

at 4 hours-3000 ng/ml

at 9 hours-1800 ng/ml

at 12 hours-2400 ng/ml

at 12 hours-1400 ng/ml

at 13 hours-1600 ng/ml

at 15 hours-2100 ng/ml

at 24 hours 1400 ng/ml

at 24 hours 2400 ng/ml

Control test 0.032 g Quinine mixed with 3 ml with water was injected into a rat:

9 hours-none detected

12 hours-420 ng/ml

16 hours-230 ng/ml

The effective amount of the prolongating compositions, based on a body weight of 65 kg.

The basic use of the prolongating compositions is to administer it at the time of administering the active drug. This provides the prolongating effect. The active drug and the prolongating compositions can be administered in a variety of conventional ways, viz, I. V., I. M., oral, etc.

The present invention is applicable to the full range of sulfa drugs which are otherwise known as sulfonamides having bacteriostatic properties using the prolongation solution of Example 2.

The cinchona alkaloids are a known class of antimalarial drugs. This invention is applicable to all of the antimalarial cinchona alkaloids including quinine sulfate, quinine dihydrochloride and quinacrine and its salts, known to those skilled in the art, the antimalarial cinchona alkaloids are often given in conjunction with other drugs such as pyrimethamine, the sulfonamides and sulfones. All such combinations are contemplated by the present invention.

This invention also includes the prolongation in circulating serum in mammals of primaquine, decoquinate, rifampicin, verapamil, glipizide, papaverin and streptomycin, among others.

What is claimed:

1. In the method of administering rifampicin to a human subject, the improvement wherein a prolongation composition is also administered, said prolongation composition comprising:

| Pectin | 0.94 | g |
|---|---|---|
| Potassium bitartrate | 0.48 | g |
| Tannic acid | 0.8 | g |
| L-Tyrosine | 0.21 | g |
| Riboflavin | 2 ml 10% solution of 15% Ethyl Alcohol | |
| Hexanoic Acid (Caproic acid) | 0.06 | ml |
| | |
|---|---|
| Ethyl Alcohol | 15% Ethyl Alcohol to make 100 ml |
